(19) United States
(12) Patent Application Publication
(10) Pub. No.: US 2023/0167061 A1
(43) Pub. Date: Jun. 1, 2023

(54) PREPARATION OF COMPOUNDS HAVING PESTICIDAL ACTIVITY

(71) Applicant: CORTEVA AGRISCIENCE LLC, INDIANAPOLIS, IN (US)

(72) Inventors: AMARUKA HAZARI, CARMEL, IN (US); NICHOLAS M. IRVINE, WESTFIELD, IN (US); MELISSA LEE, INDIANAPOLIS, IN (US); XIAOYONG LI, ZIONSVILLE, IN (US); ABRAHAM SCHUITMAN, ZIONSVILLE, IN (US); SUELEN VASQUEZ CESPEDES, CARMEL, IN (US)

(73) Assignee: CORTEVA AGRISCIENCE LLC, INDIANAPOLIS, IN (US)

(21) Appl. No.: 17/997,675

(22) PCT Filed: May 7, 2021
(86) PCT No.: PCT/US2021/031226

§ 371 Date: Nov. 1, 2022

Related U.S. Application Data
(60) Provisional application No. 63/022,830, filed on May 11, 2020.

(51) Int. Cl. C07D 213/803; B01J 31/28

(52) U.S. Cl. CPC C07D 213/803; B01J 31/28

(57) ABSTRACT

A method of forming a compound of formula (I) is provided. Some embodiments of the compound of formula (I) are known to have herbicidal activity.

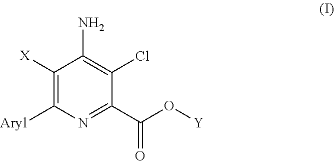

The method comprises combining a compound of formula (II) and a compound of formula (IIIa) or (IIIb), or a salt or ester thereof:

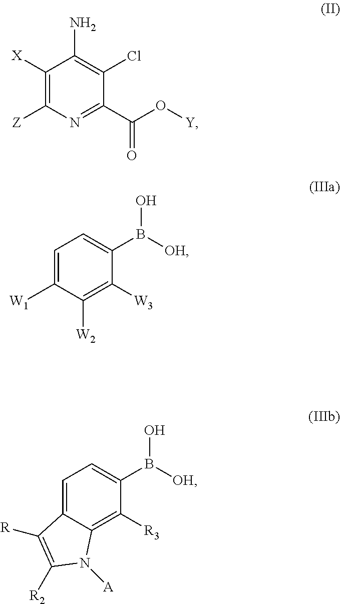

in a liquid mixture comprising the compounds of formula (II), formula (IIIa) or (IIIb), water, a non-aqueous solvent, a surfactant, a catalyst, and a ligand at certain temperature, pH, and HLB ranges to form a chemical reaction product mixture comprising the compound of formula (I) and by-products.

### BACKGROUND

Certain synthetic chemical compounds have been identified as being synthetic auxin herbicides. These synthetic chemical compounds have been demonstrated to offer control of certain weeds in many agricultural settings. While various reaction schemes exist for producing these certain synthetic auxins, new methods that utilize different reaction schemes remain of interest to those who produce these synthetic auxins. Such discoveries can lead to manufacturing processes, e.g., of improved efficiency.

### SUMMARY

A method of forming a compound of formula (I) is provided. Formula (I) is as follows:

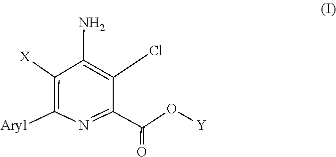

wherein X represents H, F; Y represents CH₂Ph, Me, CH₂CN, H; and Aryl represents a substituted or unsubstituted aryl or heteroaryl group. The method comprises combining a compound of formula (II) and a compound of formula (IIIa) or (IIIb), or a salt or ester thereof (e.g., lithium salt, sodium salt, potassium salt, etc.):

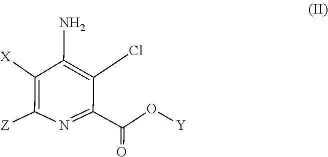

wherein X represents H, F; Y represents Me, CH₂Ph, CH₂CN, H; and Z represents Cl, Br;

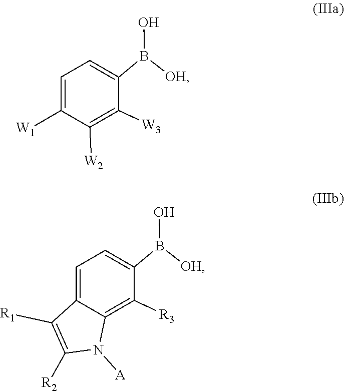

wherein W₁ represents halogen, alkyl, alkyloxy, haloalkyl, haloalkoxy, nitrile, or nitro group; W₂ represents H, F, Cl, alkyl, alkoxy, haloalkyl, haloalkoxy, or alkyl-substituted amino group; W₃ represents H, F, Cl, alkyl or alkoxy group; A represents H or a silyl alkyl group; R₁ represents H, halogen, alkyl, alkyloxy, haloalkyl, haloalkoxy, nitrile, or nitro group; R₂ represents H, halogen, alkyl, alkyloxy, haloalkyl, haloalkoxy, nitrile, or nitro group; and R₃ represents H, F, Cl, alkyl or alkoxy; in a liquid mixture comprising the compounds of formula (II), formula (IIIa) or (IIIb), or a salt or ester thereof (e.g., lithium salt, sodium salt, potassium salt, etc.), water, a non-aqueous solvent, a surfactant, a catalyst, and a ligand at from about 0 to about 70° C., a pH of from about 6 to 14 and a hydrophilic-lipophilic balance (“HLB”) of from about 9 to about 15 to form a chemical reaction product mixture comprising the compound of formula (I) and by-products.

### BRIEF DESCRIPTION OF THE DRAWING

FIG. 1 graphically illustrates percent conversion per time unit of the several examples provided herewith.

### DETAILED DESCRIPTION

A method of forming a compound of formula (I) is provided. Formula (I) is as follows:

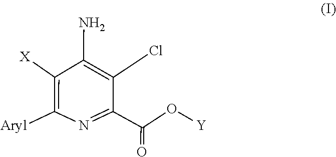

wherein X represents H, F; Y represents CH₂Ph, Me, CH₂CN, H; and Aryl represents a substituted or unsubstituted aryl or heteroaryl group. The method comprises combining a compound of formula (II) and a compound of formula (IIIa) or (IIIb), or a salt or ester thereof (e.g., lithium salt, sodium salt, potassium salt, etc.):

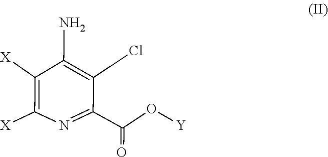

wherein X represents H, F; Y represents Me, CH₂Ph, CH₂CN, H; and Z represents Cl, Br;

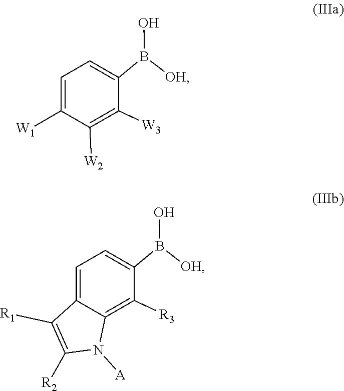

wherein W₁ represents halogen, alkyl, alkyloxy, haloalkyl, haloalkoxy, nitrile, or nitro group; W₂ represents H, F, Cl, alkyl, alkoxy, haloalkyl, haloalkoxy, or alkyl-substituted amino group; W₃ represents H, F, Cl, alkyl or alkoxy group; A represents H or a silyl alkyl group; R₁ represents H, halogen, alkyl, alkyloxy, haloalkyl, haloalkoxy, nitrile, or nitro group; R₂ represents H, halogen, alkyl, alkyloxy, haloalkyl, haloalkoxy, nitrile, or nitro group; and R₃ represents H, F, Cl, alkyl or alkoxy; in a liquid mixture comprising the compounds of formula (II), formula (IIIa) or (IIIb), or a salt or ester thereof (e.g., lithium salt, sodium salt, potassium salt, etc.), water, a non-aqueous solvent, a surfactant, a catalyst, and a ligand at from about 0 to about 70° C., a pH of from about 6 to 14 and a hydrophilic-lipophilic balance (“HLB”) of from about 9 to about 15 to form a chemical reaction product mixture comprising the compound of formula (I) and by-products.

When utilized, a compound of formula (IIIb) may comprise an unprotected or a protected amine. In other words, in the formula, when “A” represents hydrogen (“H”), the amine is said to be unprotected. When “A” represents a silyl alkyl group, the amine is said to be protected. When present, the silyl alkyl group may be straight-chain, branched, etc. In certain embodiments of the methods provided herein, the silyl alkyl group is selected from trimethylsilyl (“TMS”), tert-butyldiphenylsilyl (“TBDPS”), tert-butyldimethylsilyl (“TBS”), and triisopropylsilyl (“TIPS”) groups. In certain embodiments of the methods, the silyl alkyl group is TBS.

The liquid mixture is maintained at conditions such that the liquid mixture forms a chemical reaction product mixture comprising the compound of formula (I) and by-products. The liquid mixture should be maintained at a temperature of from about 0 to about 70° C., a pH of from about 6 to 14 and a hydrophilic-lipophilic balance (“HLB”) of from about 9 to about 15.

In certain embodiments of the method, the liquid mixture has an HLB of from about 9 to about 15. In certain embodiments of the method, the liquid mixture has an HLB of from about 9, or from about 10, or from about 11, or from about 12, to about 15, or to about 14. The endpoints of the HLB range of the liquid mixture can be any combination of the foregoing, e.g., from about 9 to about 15, or from about 10 to about 15, or from about 11 to about 15, or from about 12 to about 14, or from about 11 to about 14, or from about 9 to about 14, and so forth. In certain embodiments of the method, the HLB is about 13. The methods provided herein rely upon partitioning and shuttling reactants and reagents into and out of the micelles. While not wishing to be bound by theory, it is believed that an optimal HLB provides optimal reaction conditions in part because reactants and reaction products can be shuttled into and out of the micelles formed in the liquid mixture described herein.

In certain embodiments of the method, the liquid mixture has a pH of from about 6 to 14. In certain embodiments of the method, the liquid mixture has a pH of from at least about 6, or at least about 7, or at least about 8, to 14, or to about 13, or to about 12, or to about 11. The endpoints of the pH range of the liquid mixture can be any combination of the foregoing, e.g., from about 6 to 14, or from about 6 to about 13, or from about 7 to about 12, or from about 6 to about 11, or from about 8 to about 13, or from about 8 to about 11, or from about 8 to about 12, and so forth.

In certain embodiments of the method, the liquid mixture has a temperature of from about 0 to about 70° C. In certain embodiments of the method, the liquid mixture has a temperature of from at least about 0° C., or at least about 10° C., or at least about 20° C., or at least about 30° C., or at least about 40° C., to about 70° C., or to about 65° C., or to about 60° C., or to about 55° C., or to about 50° C. The endpoints of the temperature range of the liquid mixture can be any combination of the foregoing, e.g., from about 0° C. to about 70° C., or from about 0° C. to about 65° C., or from about 10° C. to about 70° C., or from about 20° C. to about 70° C., or from about 30° C. to about 70° C., or from about 20° C. to about 65° C., or from about 30° C. to about 65° C., or from about 30° C. to about 65° C., or from about 30° C. to about 60° C., or from about 40° C. to about 70° C., or from about 40° C. to about 65° C., or from about 40° C. to about 60° C., or from about 40° C. to about 55° C., or from about 40° C. to about 50° C., and so forth.

Generally, the methods provided herein result in a chemical reaction product mixture comprising the compound of formula (I) of suitable yield and purity. In certain embodiments of the method, the chemical reaction product mixture comprises the compound of formula (I) at a concentration of from about 1 wt % to about 22 wt %. In certain embodiments of the method, the chemical reaction product mixture comprises the compound of formula (I) at a concentration of at least about 1 wt %, or at least about 2 wt %, or at least about 3 wt %, or at least about 4 wt %, or at least about 5 wt %, to about 22 wt %, or to about 20 wt %, or to about 18 wt %, or any combination thereof. In certain embodiments of the method, the chemical reaction product mixture comprises the compound of formula (I) at a concentration of from about 5 wt % to about 20 wt %.

In certain embodiments, the method further comprises separating at least a portion of the by-products from the chemical reaction product mixture to form a purified chemical reaction product mixture and a heel comprising the catalyst. The term “heel” is utilized herein merely to describe the portion of the separated by-products from the chemical reaction product mixture that contains all or at least a substantial portion of the catalyst.

Separation of at least a portion of the by-products from the chemical reaction product mixture to form the purified chemical reaction product mixture and a heel comprising the catalyst can be carried out by any suitable separation process. For example, an organic solvent can be added to the chemical reaction product mixture leading to two liquid phases that can be separated via one or more processes known to those skilled in the art. The separated organic phase containing the product can be further purified via crystallization. The aqueous phase (e.g., heel) contains the catalyst.

In an alternative separation process, the chemical reaction product mixture is cooled to ambient temperature leading to the formation of a slurry of solids. Filtration of the slurry followed by washing with an appropriate solvent (e.g., water) can be performed to provide a purified chemical reaction product mixture (e.g., the resulting cake). The purified chemical reaction product mixture can be re-dissolved in an appropriate solvent for further purification, e.g., via crystallization if desired.

In certain embodiments of the method, the compound of formula (I) comprises from about 10 wt % to about 80 wt % of the purified chemical reaction product mixture. In certain embodiments of the method, the purified chemical reaction product mixture comprises the compound of formula (I) at a concentration of at least about 10 wt %, or at least about 20 wt %, or at least about 30 wt %, to about 80 wt %, or to about 70 wt %, or to about 60 wt %, or any combination thereof. In certain embodiments of the method, the purified chemical reaction product mixture comprises the compound of formula (I) at a concentration of from about 10 wt % to about 80 wt %, or from about 20 wt % to about 70 wt %, or from about 30 wt % to about 60 wt %, or from about 10 wt % to about 60 wt %, or from about 20 wt % to about 80 wt %, or from about 30 wt % to about 70 wt %, and so forth.

In certain embodiments of the method, the compound of formula (I) is

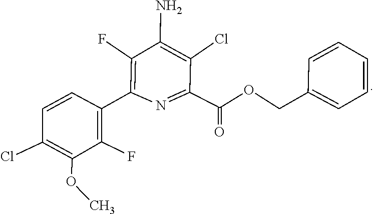 [SMILES: COc1c(Cl)ccc(-c2nc(C(=O)OCc3ccccc3)c(Cl)c(N)c2F)c1F]

In certain embodiments of the method, the compound of formula (II) is

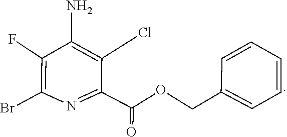 [SMILES: Nc1c(F)c(Br)nc(C(=O)OCc2ccccc2)c1Cl]

The liquid mixture of the methods provided herein are multiphase in that the liquid mixture includes each of an aqueous solvent (e.g., water) and a non-aqueous solvent. The non-aqueous solvent can be any suitable non-aqueous solvent for carrying out the methods provided herewith. In certain embodiments of the method, the non-aqueous solvent is selected from tetrahydrofuran (“THF”), acetone, acetonitrile, ethyl acetate, methyl ethyl ketone (“MEK”), methyl isobutyl ketone (“MIBK”), methanol, ethanol, isopropanol, polyethyleneglycol (“PEG”), or a combination thereof. In certain embodiments, the non-aqueous solvent is THF.

The surfactant may be any suitable surfactant for carrying out the methods provided herewith. The surfactant allows for interaction between the aqueous phase of the liquid mixture and the non-aqueous phase of the liquid mixture via micelles. In certain embodiments of the method, the surfactant is a cationic surfactant, a nonionic surfactant, or a combination thereof.

In certain embodiments of the method, the surfactant is a cationic surfactant. A cationic surfactant is a surfactant having a net positive charge, and often is a polymeric compound. Exemplary embodiments of cationic monomer units that can be utilized to form cationic surfactants include but are not limited to allyl amine, vinyl amine, dialkylaminoalkyl acrylates and methacrylates and their quaternary or acid salts, including, but not limited to, dimethylaminoethyl acrylate methyl chloride quaternary salt (“DMAEA.MCQ”), dimethylaminoethyl acrylate methyl sulfate quaternary salt, dimethyaminoethyl acrylate benzyl chloride quaternary salt, dimethylaminoethyl acrylate sulfuric acid salt, dimethylaminoethyl acrylate hydrochloric acid salt, dimethylaminoethyl methacrylate methyl chloride quaternary salt, dimethylaminoethyl methacrylate methyl sulfate quaternary salt, dimethylaminoethyl methacrylate benzyl chloride quaternary salt, dimethylaminoethyl methacrylate sulfuric acid salt, dimethylaminoethyl methacrylate hydrochloric acid salt, dialkylaminoalkylacrylamides or methacrylamides and their quaternary or acid salts such as acrylamidopropyltrimethylammonium chloride, dimethylaminopropyl acrylamide methyl sulfate quaternary salt, dimethylaminopropyl acrylamide sulfuric acid salt, dimethylaminopropyl acrylamide hydrochloric acid salt, methacrylamidopropyltrimethylammonium chloride, dimethylaminopropyl methacrylamide methyl sulfate quaternary salt, dimethylaminopropyl methacrylamide sulfuric acid salt, dimethylaminopropyl methacrylamide hydrochloric acid salt, diethylaminoethylacrylate, diethylaminoethylmethacrylate, diallyldiethylammonium chloride, and diallyldimethyl ammonium chloride (“DADMAC”).

In certain embodiments of the method, the surfactant is a nonionic surfactant. A nonionic surfactant is a surfactant having no charge. Nonionic surfactants generally are polymeric compounds. Exemplary embodiments of nonionic monomer units that can be combined to form nonionic surfactants include but are not limited to acrylamide, methacrylamide, N,N-dimethylacrylamide, N,N-diethylacrylamide, N-isopropylacrylamide, N-vinylformamide, N-vinylmethylacetamide, N-vinyl pyrrolidone, hydroxyethyl methacrylate, hydroxyethyl acrylate, hydroxypropyl acrylate, hydroxypropyl methacrylate, N-t-butylacrylamide, N-methylolacrylamide, vinyl acetate, and vinyl alcohol.

In certain embodiments of the method, the nonionic surfactant is a polymer comprising polyethylene glycol ethers capped at one end with a long aliphatic chain. For example, the aliphatic chain may comprise, but is not limited to, a linear or branched alkyl group bearing 8-20 carbon atoms.

Additionally, other nonionic surfactants have shown usefulness in the methods provided herein. In certain embodiments of the method, the surfactant is a nonionic surfactant having the formula of A-K-B, wherein A is a hydrophobic head, B is a hydrophilic tail, and K is a linker, and in certain embodiments, A is tocopherol, B is polyethylene glycol 750 (“PEG-750”), and K is a dicarboxylic acid, e.g., succinic acid. In certain embodiments of the method, the surfactant is a nonionic surfactant, and the nonionic surfactant is tocopherol polyethylene glycol 750-Methyl succinate (“TPGS-750” or “TPGS-750-Me”).

The liquid mixture of the methods provided herewith comprises a catalyst. The catalyst may be any catalyst suitable for use to react a halogenated aromatic or halogenated heteroaromatic compound with an aromatic boronic acid, salt, or ester, e.g., a compound of formula (II) with a compound of formula (IIIa) or (IIIb) to form a compound of formula (I). In certain embodiments of the method, the catalyst is a palladium-based catalyst. In certain embodiments of the method, the palladium-based catalyst is selected from an organic palladium compound and an inorganic palladium compound. In certain embodiments of the method the catalyst is palladium (II) acetate.

The liquid mixture of the methods provided herewith comprises a ligand. The ligand is used to complex with the catalyst to increase catalyst activity and stabilize the precious metal species. In certain embodiments of the method, the ligand is an organophosphorus compound, which in certain embodiments is selected from a monodentate organophosphine or bidentate organophosphine. In certain embodiments of the methods, ligand is an organophosphorus compound, which is triphenylphosphine.

Generally, to maintain pH at a preferred range, the liquid mixture may further comprise a buffer. In certain embodiments of the method, the liquid mixture further comprises a buffer. When utilized, the buffer may be any suitable buffer so as to facilitate the reaction of the compound of formula (II) and the compound of formula (IIIa) or (IIIb), or a salt or ester thereof (e.g., lithium salt, sodium salt, potassium salt, etc.).

When present, the buffer may be, e.g., an inorganic salt and/or an organic amine base. In certain embodiments of the method, the liquid mixture comprises a buffer selected from an inorganic salt, an organic amine base, or a combination thereof. Examples of suitable inorganic salts include a metal bicarbonate (e.g., sodium bicarbonate, potassium bicarbonate, etc.), a metal carbonate (e.g., sodium carbonate, potassium carbonate, etc.), a metal phosphate (e.g., sodium phosphate, potassium phosphate, etc.), a metal fluoride (e.g., sodium fluoride, potassium fluoride, etc.), or a combination thereof. In certain embodiments of the method, the liquid mixture comprises a buffer selected from sodium bicarbonate, potassium bicarbonate, or a combination thereof. In certain embodiments of the method, the liquid mixture comprises a buffer, the buffer being potassium bicarbonate.

In certain embodiments of the method, the liquid mixture comprises a buffer that is an organic amine base. In certain embodiments of the method, the liquid mixture includes a buffer that is an organic amine base selected from triethyl amine, diisopropylethylamine, picoline, or a combination thereof.

### EMBODIMENTS

The invention is further illustrated by the following embodiments.

(1) A method of forming a compound of formula (I) as described herein is provided. The method comprises combining compounds of formula (II) and either (IIIa) or (IIIb), or a salt, ester, or ether thereof (e.g., lithium salt, sodium salt, potassium salt), also as described herein, in a liquid mixture comprising the compounds of formula (II) and (IIIa) or (IIIb), or a salt or ester thereof (e.g., lithium salt, sodium salt, potassium salt, etc.), water, a non-aqueous solvent, a surfactant, a catalyst, and a ligand at 0-70° C. and a pH of from about 6 to 14 and a hydrophilic-lipophilic balance (“HLB”) of from about 9 to about 15 to form a chemical reaction product mixture comprising the compound of formula (I) and by-products.

(2) The method of embodiment (1), wherein the chemical reaction product mixture comprises the compound of formula (I) at a concentration of from about 5 wt % to about 20 wt %.

(3) The method of embodiment (1) or (2), wherein the non-aqueous solvent is selected from tetrahydrofuran (“THF”), acetone, acetonitrile, ethyl acetate, methyl ethyl ketone (“MEK”), methyl isobutyl ketone (“MIBK”), methanol, ethanol, isopropanol, polyethyleneglycol (“PEG”), or a combination thereof.

(4) The method of any one of embodiments (1)-(3), wherein the liquid mixture has an HLB of from about 12 to about 14.

(5) The method of any one of embodiments (1)-(4), wherein the surfactant is selected from a cationic surfactant, a nonionic surfactant, or a combination thereof.

(6) The method of any one of embodiments (1)-(5), wherein the surfactant is a nonionic surfactant.

(7) The method of embodiment (6), wherein the nonionic surfactant has the formula of A-K-B, wherein A is a hydrophobic head, B is a hydrophilic tail, and K is a linker.

(8) The method of embodiment (7), wherein A is tocopherol, B is PEG-750-Me, and K is a dicarboxylic acid.

(9) The method of embodiment (8), wherein the dicarboxylic acid is succinic acid.

(10) The method of any one of embodiments (1)-(9), wherein the palladium-based catalyst is selected from an organic palladium compound and an inorganic palladium compound.

(11) The method of embodiment (10), wherein the palladium-based catalyst is palladium (II) acetate.

(12) The method of any one of embodiments (1)-(11), wherein the ligand is an organophosphorus compound.

(13) The method of embodiment (12), wherein the organophosphorus compound is selected from a monodentate organophosphine or bidentate organophosphine.

(14) The method of embodiment (12), wherein the organophosphorus compound is triphenylphosphine.

(15) The method of any one of embodiments (1)-(14), wherein the liquid mixture further comprises a buffer.

(16) The method of embodiment (15), wherein the buffer is selected from an inorganic salt and an organic amine base.

(17) The method of embodiment (16), wherein the buffer is an inorganic salt selected from a metal bicarbonate, a metal carbonate, a metal phosphate, a metal fluoride, or a combination thereof.

(18) The method of any one of embodiments (15)-(17), wherein the buffer is a metal bicarbonate selected from sodium bicarbonate and potassium bicarbonate.

(19) The method of embodiment (18), wherein the buffer is potassium bicarbonate.

(20) The method of embodiment (16), wherein the buffer is an organic amine base selected from triethyl amine, diisopropylethylamine, picoline, or a combination thereof.

(21) The method of any one of embodiments (1)-(20), wherein the compound of formula (I) is

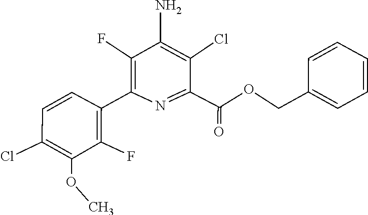 [SMILES: COc1c(Cl)ccc(-c2nc(C(=O)OCc3ccccc3)c(Cl)c(N)c2F)c1F]

(22) The method of any one of embodiments (1)-(21), wherein the compound of formula (II) is

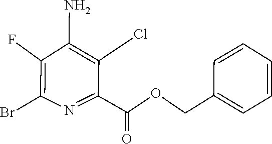 [SMILES: Nc1c(F)c(Br)nc(C(=O)OCc2ccccc2)c1Cl]

(23) The method of any one of embodiments (1)-(22), further comprising separating at least a portion of the by-products from the chemical reaction product mixture to form a purified chemical reaction product mixture and a heel comprising the catalyst.

### EXAMPLES

### Control Example

In a glovebox under nitrogen atmosphere, a 20-mL vial equipped with a magnetic stir bar was charged palladium (II) acetate (Strem, Lot: 34487200, 4.5 mg, 0.02 mmol, 0.005 eq.), triphenylphosphine (Aldrich, 10.5 mg, 0.04 mmol, 0.010 eq.), a compound of formula (IIIa), wherein W₁=Cl, W₂=OMe, and W₃=F (Lianhe, Lot: XLT-PBA201604090, 856 mg, 4.20 mmol, 1.05 eq.), a compound of formula (II), wherein X=F, Y=CH₂Ph, and Z=Br (ENBK-170037-79-8, 96.5%, 1.452 g, 4.0 mmol, 1.0 eq.), acid form, potassium bicarbonate (Aldrich, 98%, 800 mg, 8.0 mmol, 2.0 eq.), THF (Aldrich, 99%, 1.8 mL), and water (7.2 mL), without surfactant leading to a liquid mixture in the form of a milky slurry. The liquid mixture was stirred at 50° C. at 500 rpm on a stir plate and monitored by liquid chromatography (“LC”). The reaction was stopped after 30 h when LC indicated approximately 63% conversion. Light brown solids precipitated upon cooling to room temperature. The solid were filtered and washed with water (3.0 mL). The wet cake (2.253 g) was dissolved in THF (3.164 g) and assayed by LC which indicated 56% yield of florpyrauxifen-benzyl.

### Example 1

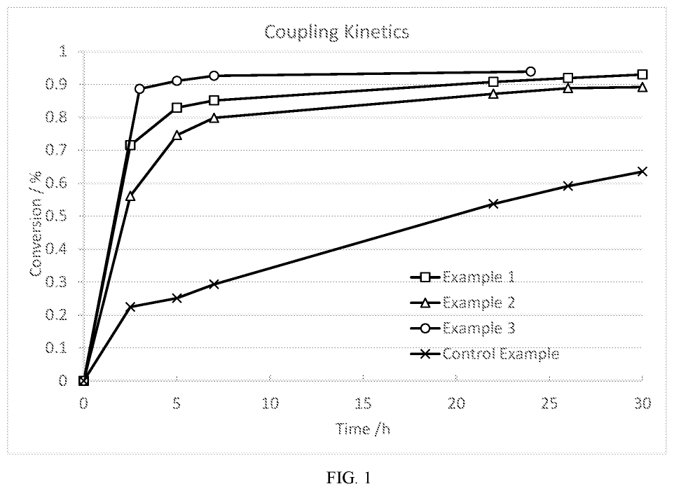

In a glovebox under nitrogen atmosphere, a 20-mL vial equipped with a magnetic stir bar was charged palladium (II) acetate (Strem, Lot: 34487200, 4.5 mg, 0.02 mmol, 0.005 eq.), triphenylphosphine (Aldrich, 10.5 mg, 0.04 mmol, 0.010 eq.), a compound of formula (IIIa), wherein W₁=Cl, W₂=OMe, and W₃=F (Lianhe, Lot: XLT-PBA201604090, 856 mg, 4.20 mmol, 1.05 eq.), acid form, a compound of formula (II), wherein X=F, Y=CH₂Ph, and Z=Br (ENBK-170037-79-8, 96.5%, 1.452 g, 4.0 mmol, 1.0 eq.), potassium bicarbonate (Aldrich, 98%, 800 mg, 8.0 mmol, 2.0 eq.), THF (Aldrich, 99%, 1.8 mL) and 2 wt % TPGS-750-M (7.2 mL) leading to a liquid mixture in the form of a milky slurry. The liquid mixture was stirred at 50° C. at 500 rpm on a stir plate and monitored by LC. The reaction was stopped after 30 h when LC indicated 93.0% conversion. Two distinct liquid layers observed upon settling. Light brown solids precipitated upon cooling to room temperature. The solid were filtered and washed with water (3.0 mL). The wet cake (2.791 g) was dissolved in THF (3.735 g) and assayed by LC which indicated 80% yield of florpyrauxifen-benzyl. Compared to the control, this mixture included surfactant (e.g., TPGS-750-Me). As can be seen in FIG. 1, the reaction of Example 1 (as well as Examples 2 and 3) progressed more quickly than the control reaction.

### Example 2

In a glovebox under nitrogen atmosphere, a 20-mL vial equipped with a magnetic stir bar was charged palladium (II) acetate (Strem, Lot: 34487200, 2.3 mg, 0.01 mmol, 0.0025 eq.), triphenylphosphine (Aldrich, 10.5 mg, 0.04 mmol, 0.010 eq.), a compound of formula (IIIa), wherein W₁=Cl, W₂=OMe, and W₃=F (Lianhe, Lot: XLT-PBA201604090, 856 mg, 4.20 mmol, 1.05 eq.), acid form, a compound of formula (II), wherein X=F, Y=CH₂Ph, and Z=Br (ENBK-170037-79-8, 96.5%, 1.452 g, 4.0 mmol, 1.0 eq.), potassium bicarbonate (Aldrich, 98%, 800 mg, 8.0 mmol, 2.0 eq.), THF (Aldrich, 99%, 1.8 mL) and 2 wt % TPGS-750-M (7.2 mL) leading to a liquid mixture in the form of a milky slurry. The liquid mixture was stirred at 50° C. at 500 rpm on a stir plate and monitored by LC. The reaction was stopped after 30 h when LC indicated 89.2% conversion. Two distinct liquid layers observed upon settling. Light brown solids precipitated upon cooling to room temperature. The solid were filtered and washed with water (3.0 mL). The wet cake (2.336 g) was dissolved in THF (4.226 g) and assayed by LC which indicated 74% yield of florpyrauxifen-benzyl.

### Example 3

In a glovebox under nitrogen atmosphere, a 20-mL vial equipped with a magnetic stir bar was charged palladium (II) acetate (Strem, Lot: 34487200, 2.3 mg, 0.01 mmol, 0.005 eq.), triphenylphosphine (Aldrich, 5.3 mg, 0.02 mmol, 0.01 eq.), a compound of formula (IIIa), wherein W₁=Cl, W₂=OMe, and W₃=F (Lianhe, Lot: XLT-PBA201604090, 470 mg, 2.3 mmol, 1.15 eq.), acid form, a compound of formula (II), wherein X=F, Y=CH₂Ph, and Z=Br (ENBK-170037-79-8, 96.5%, 0.719 g, 2.0 mmol, 1.0 eq.), potassium bicarbonate (Aldrich, 98%, 400 mg, 4.0 mmol, 2.0 eq.), THF (Aldrich, 99%, 0.8 mL) and 2 wt % TPGS-750-M (3.6 mL) leading to a liquid mixture in the form of a milky slurry. The liquid mixture was stirred at 60° C. at 500 rpm on a stir plate and monitored by LC. The reaction was stopped after 24 h when LC indicated 93.9% conversion. Two distinct liquid layers observed upon settling. Light brown solids precipitated upon cooling to room temperature. The solid were filtered and washed with water (2.0 mL). The wet cake (1.526 g) was dissolved in THF (3.205 g) and assayed by LC which indicated 81% yield of florpyrauxifen-benzyl. Reaction parameters for the Examples are summarized in Table I below, and results of the Examples are shown graphically in FIG. 1.

TABLE I
Summary of certain reaction parameters
of the Control and Examples 1-3.

| Entry | TPGS-750-Me | Pd loading | | III-a loading | Temperature |
|---|---|---|---|---|---|
| Control | 0 wt % | 0.005 | eq. | 1.05 eq. | 50° C. |
| Example 1 | 2 wt % | 0.005 | eq. | 1.05 eq. | 50° C. |
| Example 2 | 2 wt % | 0.0025 | eq. | 1.05 eq. | 50° C. |
| Example 3 | 2 wt % | 0.005 | eq. | 1.15 eq. | 60° C. |

### Example 4

Under ambient conditions, to a 20 ml scintillation vial equipped with a magnetic stir bar, was added palladium(II) acetate (4.49 mg, 0.020 mmol, 1 mol %), triphenylphosphine (10.49 mg, 0.040 mmol, 2 mol %), potassium carbonate (0.691 g, 5.00 mmol, 2.5 equiv), 4-amino-6-bromo-3-chloro-5-fluoropicolinic acid (BFAP, 0.539 g, 2 mmol, 1 equiv), (7-fluoro-1H-indol-6-yl)boronic acid (0.429 g, 2.400 mmol, 1.2 equiv). The vial was transferred to a nitrogen filled glovebox. To the solids were added MeCN (0.8 ml) and aqueous solution of 2% surfactant TPGS-750-M (3.2 ml). A control reaction utilized 3.2 ml water in place of the aqueous surfactant. The reactions were heated at 60° C. overnight. After 24 h, the aqueous phase was analyzed by quantitative HPLC with dimethylphathalate as the internal standard. A compound of Formula (I) was generated, wherein X represents F and Y represents H.

| | | | BFAP (% | |
|---|---|---|---|---|
| substrate | conditions | Base | unreacted) | Yield % |
| BFAP | water/MeCN | K₂CO₃ | 78 | 9 |
| BFAP | 2% TPGS-750- | K₂CO₃ | 52 | 34 |
| | M/MeCN | | | |

### Example 5

Under ambient conditions, to a 20 ml scintillation vial equipped with a magnetic stir bar, was added palladium(II) acetate (4.49 mg, 0.020 mmol, 1 mol %), triphenylphosphine (10.49 mg, 0.040 mmol, 2 mol %), potassium carbonate (0.691 g, 5.00 mmol, 2.5 equiv), benzyl 4-amino-6-bromo-3-chloro-5-fluoropicolinate (BFAP-Bn, 0.719 g, 2 mmol, 1 equiv), (7-fluoro-1H-indol-6-yl)boronic acid (0.429 g, 2.400 mmol, 1.2 equiv). The vial was transferred to a nitrogen filled glovebox. To the solids were added THF or MeCN (0.8 ml) and aqueous solution of 2% TPGS-750-M (3.2 ml). The control reaction utilized 3.2 ml water in place of the aqueous surfactant. The reactions were heated at 60° C. overnight. After 24 h, THF (THF) was added to the reactions. The organic phase was separated and analyzed by quantitative ¹⁹F NMR with bis(4-fluorophenyl)methanone as the internal standard to determine the mass of product and the remaining starting material. A compound of Formula (I) was generated, wherein X represents F and Y represents benzyl.

| | | | BFAP-Bn (% | 263-Bn |
|---|---|---|---|---|
| substrate | conditions | Base | unreacted) | Yield % |
| BFAP-Bn | water/MeCN | K₂CO₃ | 26 | 40 |
| BFAP-Bn | 2% TPGS-750- | K₂CO₃ | 10 | 74 |
| | M/MeCN | | | |
| BFAP-Bn | water/THF | K₂CO₃ | 10 | 70 |
| BFAP-Bn | 2% TPGS-750- | K₂CO₃ | 10 | 71 |
| | M/THF | | | |
| BFAP-Bn | 2% TPGS-750- | KHCO₃ | 28 | 58 |
| | M/THF | | | |
| BFAP-Bn | 2% TPGS-750- | KF | 23 | 60 |
| | M/THF | | | |

### Example 6

In a glovebox under nitrogen atmosphere, a 20-mL vial was equipped with a magnetic stir bar and charged with palladium (II) acetate (4.50 mg, 0.02 mmol, 0.010 eq.), triphenylphosphine (Aldrich, 10.5 mg, 0.02 mmol, 0.020 eq.), a compound of formula (II), wherein X=H, Y=Me, and Z=Cl (442 mg, 2.0 mmol, 1.0 eq.), a compound of formula (IIIa), wherein W₁=Cl, W₂=OMe, and W₃=F (469 mg, 2.3 mmol, 1.15 eq.), potassium fluoride (99%, 290 mg, 5.0 mmol, 2.50 eq.), THF (Aldrich, 99%, 0.80 mL) and 2 wt % TPGS-750-M (3.20 mL) aqueous solution. The mixture was heated to 50° C. leading to a brown milky slurry and was stirred at 500 rpm on a stir plate and monitored by LC. The reaction reached 95.2% conversion by LC at 254 nm after 26 h. The mixture was then heated to 60° C. and stirred for another 8 h. LC indicated 98.4% conversion. The biphasic mixture was cooled to ambient and leading to a pale brown solid slurry. The solid were filtered and the wet cake was washed with water (2×2.0 mL). The wet cake was dried in vacuum oven for 4 h affording the crude product as light brown solids (798 mg). LC assay indicated 88% yield of halauxifen methyl.

### Example 7

In a glovebox under nitrogen atmosphere, a 20-mL vial was equipped with a magnetic stir bar and charged with palladium (II) acetate (4.50 mg, 0.02 mmol, 0.010 eq.), triphenylphosphine (Aldrich, 10.5 mg, 0.02 mmol, 0.020 eq.), a compound of formula (II), wherein X=H, Y=Me, and Z=Cl (442 mg, 2.0 mmol, 1.0 eq.), a compound of formula (IIIa), wherein W₁=Cl, W₂=OMe, and W₃=F (469 mg, 2.3 mmol, 1.15 eq.), potassium fluoride (99%, 290 mg, 5.0 mmol, 2.50 eq.), THF (Aldrich, 99%, 0.80 mL) and 2 wt % Brij 30 (3.20 mL) aqueous solution. The mixture was heated to 60° C. leading to a brown milky slurry and was stirred at 500 rpm on a stir plate and monitored by LC. The reaction reached 92.8% conversion by LC at 254 nm after 24 h. The biphasic mixture was cooled to ambient and leading to a pale brown solid slurry. The solid were filtered and the wet cake was washed with water (2×2.0 mL). The wet cake was dried in vacuum oven for 4 h affording the crude product as light brown solids (833 mg). LC assay indicated 83% yield of halauxifen methyl.

### Control Example

In a glovebox under nitrogen atmosphere, a 20-mL vial was equipped with a magnetic stir bar and charged with palladium (II) acetate (4.50 mg, 0.02 mmol, 0.010 eq.), triphenylphosphine (Aldrich, 10.5 mg, 0.02 mmol, 0.020 eq.), a compound of formula (II), wherein X=H, Y=Me, and Z=Cl (442 mg, 2.0 mmol, 1.0 eq.), a compound of formula (IIIa), wherein W₁=Cl, W₂=OMe, and W₃=F (469 mg, 2.3 mmol, 1.15 eq.), potassium fluoride (99%, 290 mg, 5.0 mmol, 2.50 eq.), THF (Aldrich, 99%, 0.80 mL) and DI water (3.20 mL) aqueous solution. The mixture was heated to 60° C. leading to a dark brown milky slurry and was stirred at 500 rpm on a stir plate and monitored by LC. The reaction reached 81.2% conversion by LC at 254 nm after 24 h. The dark brown biphasic mixture was cooled to ambient.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of forming a compound of formula (I) 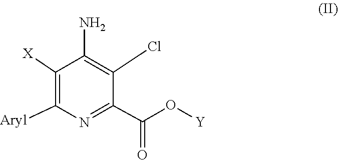
wherein X represents H, F,
Y represents CH₂Ph, Me, CH₂CN, H, and
Aryl represents a substituted or unsubstituted aryl or heteroaryl group;
comprising combining a compound of formula (II) and a compound of formula (IIIa) or (IIIb), or a salt or ester thereof, 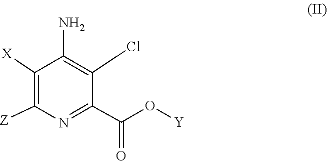
wherein X represents H, F,
Y represents Me, CH₂Ph, CH₂CN, H, and
Z represents Cl, Br; 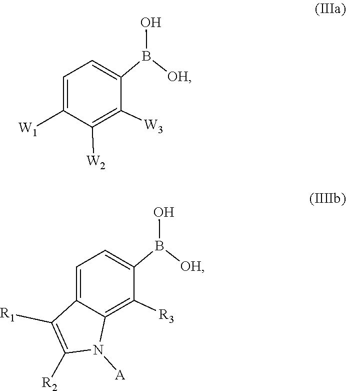
wherein W₁ represents halogen, alkyl, alkyloxy, haloalkyl, haloalkoxy, nitrile, or nitro group,
W₂ represents H, F, Cl, alkyl, alkoxy, haloalkyl, haloalkoxy, or alkylsubstituted amino group,
W₃ represents H, F, Cl, alkyl or alkoxy group,
A represents H or a silyl alkyl group,
R₁ represents H, halogen, alkyl, alkyloxy, haloalkyl, haloalkoxy, nitrile, or nitro group,
R₂ represents H, halogen, alkyl, alkyloxy, haloalkyl, haloalkoxy, nitrile, or nitro group, and
R₃ represents H, F, Cl, alkyl or alkoxy group;
in a liquid mixture comprising the compounds of formula (II) and (IIIa) or (IIIb), or a salt or ester thereof, water, a non-aqueous solvent, a surfactant, a catalyst, and a ligand at 0-70° C. and a pH of from about 6 to 14 and a hydrophilic-lipophilic balance (“HLB”) of from about 9 to about 15 to form a chemical reaction product mixture comprising the compound of formula (I) and by-products.

2. The method of claim 1, wherein the chemical reaction product mixture comprises the compound of formula (I) at a concentration of from about 5 wt % to about 20 wt %.

3. The method of claim 1, wherein the non-aqueous solvent is selected from tetrahydrofuran (“THF”), acetone, acetonitrile, ethyl acetate, methyl ethyl ketone (“MEK”), methyl isobutyl ketone (“MIBK”), methanol, ethanol, isopropanol, polyethyleneglycol (“PEG”), or a combination thereof.

4. The method of claim 1, wherein the liquid mixture has an HLB of from about 12 to about 14.

5. The method of claim 1, wherein the surfactant is selected from a cationic surfactant, a nonionic surfactant, or a combination thereof.

6. The method of claim 1, wherein the surfactant is a nonionic surfactant.

7. The method of claim 6, wherein the nonionic surfactant has the formula of A-K-B, wherein A is a hydrophobic head, B is a hydrophilic tail, and K is a linker.

8. The method of claim 7, wherein A is tocopherol, B is PEG-750-Me, and K is a dicarboxylic acid.

9. The method of claim 8, wherein the dicarboxylic acid is succinic acid.

10. The method of claim 1, wherein the palladium-based catalyst is selected from an organic palladium compound and an inorganic palladium compound.

11. The method of claim 1, wherein the palladium-based catalyst is palladium (II) acetate.

12. The method of claim 1, wherein the ligand is an organophosphorus compound.

13. The method of claim 12, wherein the organophosphorus compound is selected from a monodentate organophosphine or a bidentate organophosphine.

14. The method of claim 12, wherein the organophosphorus compound is triphenylphosphine.

15. The method of claim 1, wherein the liquid mixture further comprises a buffer.

16. The method of claim 15, wherein the buffer is selected from an inorganic salt and an organic amine base.

17. The method of claim 16, wherein the buffer is an inorganic salt selected from a metal bicarbonate, a metal carbonate, a metal phosphate, a metal fluoride, or a combination thereof.

18. The method of claim 17, wherein the inorganic salt is a metal bicarbonate selected from sodium bicarbonate or potassium bicarbonate.

19. The method of claim 18, wherein the metal bicarbonate is potassium bicarbonate.

20. The method of claim 16, wherein the buffer is an organic amine base selected from triethyl amine, diisopropylethylamine, picoline, or a combination thereof.

21. The method of claim 1, wherein the compound of formula (I) is

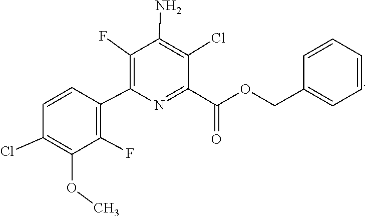 [SMILES: COc1c(Cl)ccc(-c2nc(C(=O)OCc3ccccc3)c(Cl)c(N)c2F)c1F]

22. The method of claim 1, wherein the compound of formula (II) is

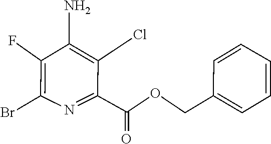 [SMILES: Nc1c(F)c(Br)nc(C(=O)OCc2ccccc2)c1Cl]

23. The method of claim 1, further comprising separating at least a portion of the by-products from the chemical reaction product mixture to form a purified chemical reaction product mixture and a heel comprising the catalyst.
